(19) United States
(12) Patent Application Publication
(10) Pub. No.: US 2026/0251158 A1
(43) Pub. Date: Aug. 27, 2026

(54) NOISE ATTENUATION FOR NUCLEAR IMAGING SYSTEM

(71) Applicant: Siemens Medical Solutions USA, Inc., Malvern, PA (US)

(72) Inventors: Christopher McBryde, Knoxville, TN (US); Kellen John Glasscock, Kingsport, TN (US); John Keller, Knoxville, TN (US)

(21) Appl. No.: 19/160,255

(22) PCT Filed: Jul. 7, 2023
(86) PCT No.: PCT/US2023/069743

§ 371 Date: Aug. 27, 2025

(51) Int. Cl. F04D 29/66; A61B 6/03; F04D 17/08; F04D 19/00

(52) U.S. Cl. CPC F04D 29/664; F04D 17/08; F04D 19/002; A61B 6/037

(57) ABSTRACT

Provided is a cooling fan assembly for a nuclear imaging system where the fan assembly can include a fan, an air inlet opening, an air outlet opening, and a honeycomb structured sheet mounted across the air inlet opening, whereby when the fan is in operation and air is flowing through the air inlet opening, the honeycomb structured sheet facilitates laminar flow of the air at the air inlet opening and attenuates acoustic noise generated by the fan.

### FIELD

The present disclosure relates generally to the field of nuclear imaging systems and, more particularly, to a technique for reducing cooling fan noise in such systems.

### BACKGROUND

In medical imaging systems in which the patient is positioned within a tunnel, such as PET and PET/CT scanners, require cooling of the associated electronics that is typically accomplished with application of forced cooling air. The application of the cooling air in such systems generally require one or more fans and the associated ducting incorporated into the patient tunnel structure to route the cooling air to where it is needed. The forced cooling air moving through such systems generate substantial level of audible noise, and when combined with the proximity of the patient tunnel structure to the patient within the imaging volume, the deleterious effect of the noise from the cooling system on the patient comfort becomes an important consideration in the design of such medical imaging systems. For example, in the existing PET and PET/CT scanner systems, the noise generated by the cooling system can be undesirably loud.

In the conventional PET or PET/CT scanners, a cooling fan or multiple cooling fans are packaged within the gantry that houses the gamma detectors to ensure the performance of the gamma detectors and other electronics therein. This assurance, however, comes with a cost. Each cooling fan pushes the noise level towards a maximum threshold and if an array of gamma detectors is to work in unison, any detector not working properly, such as by a malfunctioning fan, brings the entire system down. With large number of fan units, the statistical likelihood of a system failure is stacked against the assurance of a reliable design. Thus, a system incorporating multiple cooling fans has hidden costs associated with system downtime, service, component replacement, and patient discomfort due to noise. Conversely, distributing the cooling airflow using just one or a few fans equally around the PET gantry can be difficult to realize when accounting for the loss of gantry volume, especially as the airflow must be balanced and stable. The cost of a liquid cooling system is even higher, with failure leading to catastrophic results. Therefore, an improved cooling system for PET or PET/CT scanner.

Thus, an improved means of attenuating the noise generated by the cooling system is desired.

### SUMMARY

The present disclosure provides a nuclear imaging system comprising a cooling fan assembly that includes a fan, an air inlet opening, and a honeycomb structured sheet mounted across the air inlet opening, whereby when the fan is in operation and air is flowing through the air inlet opening, the honeycomb structured sheet facilitates laminar flow of the air at the air inlet opening and attenuates acoustic noise generated by the fan.

Also disclosed is a cooling fan assembly that includes a fan, an air inlet opening, and a honeycomb structured sheet mounted across the air inlet opening, whereby when the fan is in operation and air is flowing through the air inlet opening, the honeycomb structured sheet facilitates laminar flow of the air at the air inlet opening and attenuates acoustic noise generated by the fan.

### BRIEF DESCRIPTION OF THE DRAWINGS

The features of the embodiments described herein will be more fully disclosed in the following detailed description, which is to be considered together with the accompanying drawings wherein like numbers refer to like parts.

FIGS. 1 and 2 are cross-sectional views of a cooling fan assembly for a nuclear imaging system according to an embodiment of the present disclosure.

FIG. 3 is a view of a honeycomb structured sheet that is mounted across an air inlet opening according to an embodiment of the present disclosure.

FIG. 4 is an illustration showing a nuclear imaging system of the present disclosure.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship.

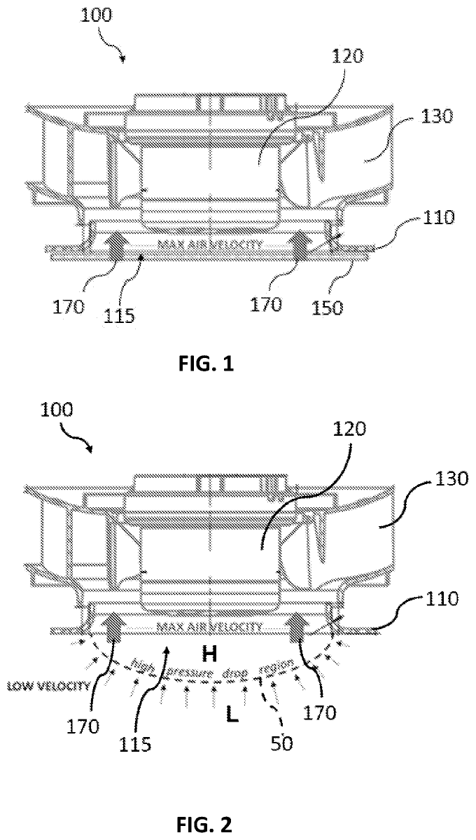

Referring to FIG. 1, a cooling fan assembly 100 for a nuclear imaging system is disclosed that incorporates a honeycomb structure in a sheet form. The fan assembly 100 comprises a fan 120, an air inlet opening 115, and a honeycomb structured sheet 150 mounted across the air inlet opening 115. In the illustrated example, an inlet ring 110 defines the air inlet opening 115. The cooling fan assembly 100 includes a housing 130 that holds the fan 120.

When the fan is in operation and air is flowing through the air inlet opening (represented by the arrows 170), the honeycomb structured sheet facilitates laminar flow of the air at the air inlet opening and attenuates acoustic noise generated by the fan. The honeycomb structured sheet attenuates acoustic noise because the honeycomb structure absorbs sound waves by being mounted in a high air velocity region. In some preferred embodiments, the honeycomb structured sheet is formed of aramid fiber. An example of such honeycomb structured sheet is Kevlar® honeycomb.

The honeycomb structured sheet formed of aramid fiber is a light weight material that is also flexible. Thus, the material is easy to work with when installing in a cooling fan assembly of a nuclear imaging system into the disclosed novel configuration that substantially lowers the noise level. Because the aramid fiber based honeycomb structures sheets are easy to work with, existing already-installed nuclear imaging systems can be retrofitted with the disclosed novel noise attenuating configuration without much trouble.

Referring to FIG. 2, as the ambient air is drawn toward the air inlet opening 115 the air velocity increases and reaches the maximum velocity at or very close to the air inlet opening 115. Thus, the region in the proximity of the air inlet opening can be defined into a low velocity region L and a high air velocity region H. The boundary between the low velocity region L and the high air velocity region H is illustrated by the broken line 50 labeled as high pressure drop region.

In the illustrated example shown in FIG. 1, the honeycomb structured sheet is in a flat planar configuration and the honeycomb structured sheet extends across the air inlet opening 115 straight. In some embodiments, the flexible honeycomb structured sheet can be molded or otherwise formed into a bowl-like shape that substantially matches the curvature of the high pressure drop region represented by the broken line 50 shown in FIG. 2.

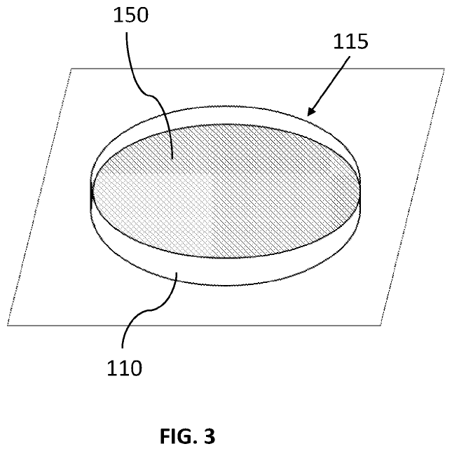

FIG. 3 shows an example of a honeycomb structured sheet 150 that is mounted across the air inlet opening 115 of a cooling fan assembly of a PET/CT scanner. Because the aramid fiber honeycomb structured sheet is flexible, the honeycomb structures sheet can be cut into an appropriate size in relation to the air inlet opening 115, inserted through the air inlet opening, allowed to expand across the air inlet opening, then mounted to the frame of the air inlet opening by an appropriate fastening means known in the art. For example, the fastening means can be Velcro, adhesive, etc.

In many nuclear imaging systems such as PET/CT scanners, the fans in the cooling fan assembly are high speed centrifugal fans and the air velocity is highest at the cooling fan assembly's air inlet. Accordingly, much of the acoustic noise generated by the cooling fan assembly emanates from the air inlet region which is the high velocity air flow region. Inventors have found that the honeycomb structured sheet is more effective at attenuating noise the closer it is to the source of the noise. Therefore, in some preferred embodiments, the honeycomb structured sheet 150 is mounted near the air inlet opening. Specifically, the honeycomb structures sheet 150 is mounted across the air inlet opening 115. In this configuration, the honeycomb structures sheet facilitates laminar flow of the air entering the cooling fan assembly through the air inlet opening.

Where there are more than one air inlet opening in the cooling fan assembly, a honeycomb structured sheet is mounted across each of the air inlet openings.

The aramid fiber honeycomb structured sheets are very effective at noise reduction in this application because of their high strength and extremely high percentage of open area that allows high degree of air flow. In some embodiments, the honeycomb structured sheet has at least 90% open area so that the placement of the honeycomb structured sheet across the air inlet opening does not adversely impact the air flowrate. In some preferred embodiments, the honeycomb structured sheet has at least 97% open area.

The fan in the cooling fan assembly can be a centrifugal fan or an axial fan. In either situation, providing a honeycomb structured sheet mounted across the air inlet opening of the cooling fan assembly can substantially attenuate noise generated by the fan in operation.

In some embodiments of the cooling fan assembly, the honeycomb structured sheet covers at least 60% of the air inlet opening. In some embodiments, the honeycomb structured sheet covers at least 70% of the air inlet opening. In some embodiments, the honeycomb structured sheet covers at least 80% of the air inlet opening. In some embodiments, the honeycomb structured sheet covers at least 90% of the air inlet opening. In some embodiments, the honeycomb structured sheet covers 100% of the air inlet opening.

In many applications, the honeycomb structured sheet can be easily installed in the high velocity air flow region of the cooling fan assembly using such attachment means as Velcro fasteners. The preferred embodiments of the honeycomb structured sheet for this application comprises the following properties: flexibility; at least 90% open area; tensile strength of ≥2,000 megapascals; Young's modulus ≥68 GPa; and density ≤1.8 g/CC. In some preferred embodiments, the honeycomb structured sheet for this application comprises: flexibility; at least 97% open area; tensile strength of ≥3,000 megapascals; Young's modulus ≥100 GPa; and density ≤1.5 g/CC.

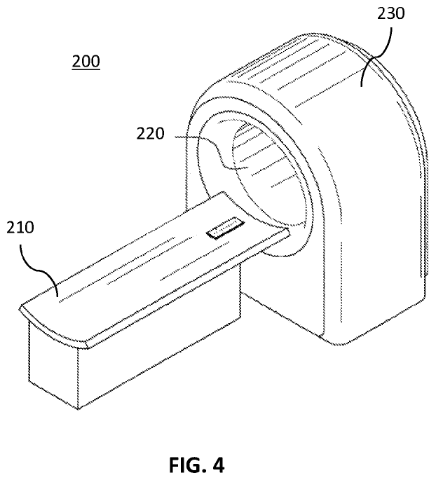

Referring to FIG. 4, also disclosed is a nuclear imaging system 200 comprising the cooling fan assembly 100 described above. The nuclear imaging system 200 comprises a patient bed 210, a scanner gantry 230, and a patient tunnel 220. One or more of the cooling fan assembly 100 are positioned in the scanner gantry 230.

It will be understood that the foregoing description is of exemplary embodiments of this invention, and that the invention is not limited to the specific forms shown. Modifications may be made in the design and arrangement of the elements without departing from the scope of the invention.

What is claimed is:

1. A cooling fan assembly for a nuclear imaging system, the fan assembly comprising:
a fan;
an air inlet opening; and
a honeycomb structured sheet mounted across the air inlet opening, whereby when the fan is in operation and air is flowing through the air inlet opening, the honeycomb structured sheet facilitates laminar flow of the air at the air inlet opening and attenuates acoustic noise generated by the fan.

2. The cooling fan assembly of claim 1, wherein the honeycomb structured sheet is formed of aramid fiber.

3. The cooling fan assembly of claim 1, wherein the honeycomb structured sheet has at least 90% open area.

4. The cooling fan assembly of claim 1, wherein the honeycomb structured sheet has at least 97% open area.

5. The cooling fan assembly of claim 1, wherein the fan is a centrifugal fan.

6. The cooling fan assembly of claim 1, wherein the fan is an axial fan.

7. The cooling fan assembly of claim 1, wherein the honeycomb structured sheet covers at least 60% of the air inlet opening.

8. The cooling fan assembly of claim 1, wherein the honeycomb structured sheet covers at least 70% of the air inlet opening.

9. The cooling fan assembly of claim 1, wherein the honeycomb structured sheet covers at least 80% of the air inlet opening.

10. The cooling fan assembly of claim 1, wherein the honeycomb structured sheet covers at least 90% of the air inlet opening.

11. The cooling fan assembly of claim 1, wherein the honeycomb structured sheet covers 100% of the air inlet opening.

12. A nuclear imaging system comprising:
a cooling fan assembly including: a fan, an air inlet opening; and
a honeycomb structured sheet mounted across the air inlet opening, whereby when the fan is in operation and air is flowing through the air inlet opening, the honeycomb structured sheet facilitates laminar flow of the air at the air inlet opening and attenuates acoustic noise generated by the fan.

13. The nuclear imaging system of claim 12, wherein the honeycomb structured sheet is formed of aramid fiber.

14. The nuclear imaging system of claim 12, wherein the honeycomb structured sheet has at least 90% open area.

15. The nuclear imaging system of claim 12, wherein the honeycomb structured sheet has at least 97% open area.

16. The nuclear imaging system of claim 12, wherein the fan is a centrifugal fan.

17. The nuclear imaging system of claim 12, wherein the fan is an axial fan.

18. The nuclear imaging system of claim 12, wherein the honeycomb structured sheet covers at least 60% of the air inlet opening.

19. The nuclear imaging system of claim 12, wherein the honeycomb structured sheet covers at least 70% of the air inlet opening.

20. The nuclear imaging system of claim 12, wherein the honeycomb structured sheet covers at least 80% of the air inlet opening.

21. The nuclear imaging system of claim 9, wherein the honeycomb structured sheet covers at least 90% of the air inlet opening.

22. The nuclear imaging system of claim 9, wherein the honeycomb structured sheet covers 100% of the air inlet opening.
